(19) United States
(12) Patent Application Publication
(10) Pub. No.: US 2002/0197338 A1
(43) Pub. Date: Dec. 26, 2002

(54) Botanical composition and methods for the treatment or prevention of obesity

(76) Inventors: Albert Peng Sheng Tseng, New South Wales (AU); Yoland Sim Huat Lim, VICTORIA (AU); Hai Ping Sun, New South Wales (AU)

Correspondence Address: Mark D. Moore, Williams, Morgan & Amerson, P.C., Suite 250, 7676 Hillmont, Houston, TX, 77040, US

(21) Appl. No.: 09/875,595

(22) Filed: Jun. 6, 2001

Related U.S. Application Data
(60) Provisional application No. 60/209,544, filed on Jun. 6, 2000.

(51) Int. Cl. A61K 35/78

(52) U.S. Cl. 424/753; 424/725

(57) ABSTRACT

Disclosed are botanical compositions, and more particularly to compositions having particularly beneficial therapeutic or prophylactic properties in a mammal. In particular, the compositions of the present inventions are useful inter alia in the treatment and/or prophylaxis of obesity, overeating, and other eating disorders, as well as an oral supplement in place of, or commensurate with, the consumption of food. The compositions of the present invention may be conveniently formulated for suitable administration, such as, for example, as a dried composition. In particularly preferred embodiments, the compositions of the present invention encompass formulations that comprise, consist essentially of, or consist of, a plurality of at least three herbal plants, horticultural and/or botanical equivalents, and/or extracts thereof, having similar or equivalent medicinal or therapeutic properties.

### BACKGROUND OF THE INVENTION

The present application claims priority from U.S. Provisional Application Serial No. 60/209,544, filed Jun. 6, 2000, the entire contents of which is specifically incorporated herein by reference in its entirety.

### FIELD OF THE INVENTION

The present invention relates generally to a composition and more particularly to a composition having therapeutic properties. The composition of the present invention is useful inter alia in the treatment and/or prophylaxis of obesity as well as an oral supplement in place of or commensurate with the consumption of food. The composition of the present invention is conveniently packaged for consumption as a dried formulation. In a particularly preferred embodiment, the composition of the present invention is a formulation derived from three or more herbal plants or horticultural and/or botanical equivalents thereof having similar or equivalent medicinal or therapeutic properties and/or extracts therefrom.

### DESCRIPTION OF RELATED ART

Obesity is one of the more significant problems frequently associated with affluent societies. Obesity is not confined to middle age and is now an increasing problem with younger children and adolescents. Although medical conditions leading to obesity exist, modem obesity frequently appears as a consequence of less exercise and an increase in the availability of fatty foods and high calorie foods at affordable prices.

Behavioural- and environmental-mediated obesity is of particular concern due to the potential ease of correcting the problem but the high cost of associated medical conditions such as heart disease and a variety of hormonal imbalance-associated conditions.

The avoidance of obesity requires a consistent and life long management approach in terms of regular exercise and control of dietary intake. However, when necessary, therapeutic intervention is an option. A variety of interventionist protocols are available including the intake of pharmacological substances. Despite some success, pharmacological substances frequently have unacceptable side affects and/or their general use is cost prohibitive.

In essence, pharmacological agents have fallen into three categories: appetite suppressants; thermogenic agents which stimulate metabolic rate thus increasing thermogenesis; and agents which regulate the size of adipose cells and the stores of lipids.

Despite the availability of pharmacological agents, safer and equally or more efficacious alternatives are required.

Traditional Chinese Medicine (TCM) is gaining greater acceptability amongst the medical profession in the Western world. One well practiced form of TCM is Herbal Chinese Medicine (HCM). The latter involves the preparation of infusions and other preparations from particular herbs. While there is certainly success with some of these formulations, the formulations themselves are prepared under relatively subjective conditions and reproducibility of the exact same conditions and/or formulations is difficult.

Modified Chinese Medicine (MCM) is becoming an important substitute to TCM. MCM requires consistency of formulation under quality-controlled environments.

In work leading up to the present invention, the inventors sought to use the concepts of MCM to prepare a composition useful in the treatment and/or prophylaxis of obesity and other weight associated conditions.

### SUMMARY OF THE INVENTION

Throughout this specification, unless the context requires otherwise, the word “comprise,” or variations such as “comprises” or “comprising,” will be understood to imply the inclusion of a stated element or integer or group of elements or integers but not the exclusion of any other element or integer or group of elements or integers.

One aspect of the present invention is directed to a composition comprising, consisting essentially of, or consisting of, an extract of herbs or horticultural or botanical equivalents of herbs or chemical or functional equivalents of the herbal extract wherein said composition is effective in the treatment or prophylaxis of obesity.

Another aspect of the present invention contemplates a composition comprising, consisting essentially of, or consisting of, at least two herbs or horticultural or botanical equivalents of said herbs, wherein said herbs are selected from those which:

(i) are useful for treating disorders affecting lipid metabolism;

(ii) have warming properties and exhibit tonic properties;

(iii) are effective in reducing blood sugar levels;

(iv) are useful in treating coronary heart disease;

(v) exhibit sedative properties; and/or

(vi) promote increased cholesterol excretion or chemical or functional equivalents of said herbal extract wherein said composition is effective in the treatment or prophylaxis of obesity.

Yet another aspect of the present invention provides a composition comprising, consisting essentially of, or consisting of, a plurality of herbs or extracts, such as, for example, at least three herbs selected from one or more species selected from the group consisting of Polygonatum sp, Nephelium sp, Hordeum sp, Ligusticum sp, Lilium sp, Rhizoma sp, Gynura sp, Companumaea sp, Codonopsis sp, and fruit of a tree, ginger essence and Juniper Q, in which one or more of said plurality of herbs:

(i) are useful for treating disorders affecting lipid metabolism;

(ii) have warming properties and exhibit tonic properties;

(iii) are effective in reducing blood sugar levels;

(iv) are useful in treating coronary heart disease;

(v) exhibit sedative properties; and/or

(vi) promote increased cholesterol excretion, or horticulture or botanical equivalent of the herb or chemical or functional equivalent of the herbal extract wherein said composition is useful in the treatment or prophylaxis of obesity.

Yet another aspect of the present invention relates to a composition useful in the treatment and/or prophylaxis of obesity and other forms of weight control, said composition comprising, consisting essentially of, or consisting of, at least two herbs or botanical or horticultural equivalents thereof, said herbs selected from Gynura pinnatifida root, Lilium brownie bulb, Nephilium longana fruit, Polygonatum multiforum root, Polygonatum canaliculatum, Condonopsis lanceolata root, Ligusticum sinense root, Hordeum vulgare, Ligusticum chuanxiong, Rhizoma zingiberis, Campanumaea lanceolata, Apple green, ginger essence and Juniper Q.

Yet another aspect of the present invention provides a composition comprising, consisting essentially of, or consisting of, maximum tolerated doses of herbs or extracts of herbs or horticultural or botanical equivalents of said herbs wherein said herbs are selected from Gynura Pinnatifida root, Lilium brownie bulb, Nephilium longana fruit, Polygonatum multiforum root, Polygonatum canaliculatum, Condonopsis lanceolata root, Ligusticum sinense root, Hordeum vulgare, Ligusticum chuanxiong, Rhizoma zingiberis, Campanumaea lanceolata, Apple green, ginger essence and Juniper Q. wherein said composition is useful for the treatment or prophylaxis of obesity.

Yet another aspect of the present invention contemplates a method for the treatment of prophylaxis of obesity in a subject, said method comprising administering to said subject an effective amount of a composition comprising two or more herbs or horticultural or botanical equivalents of said herbs wherein said herbs are selected from those which:

(i) are useful for treating disorders affecting lipid metabolism;

(ii) have warming properties and exhibit tonic properties;

(iii) are effective in reducing blood sugar levels;

(iv) are useful in treating coronary heart disease;

(v) exhibit sedative properties; and/or

(vi) promote increased cholesterol excretion; or chemical or functional equivalents of said herbal extract.

### DESCRIPTION OF ILLUSTRATIVE EMBODIMENTS

The present invention is predicated in part on the rationale design of compositions derived from herbs or other plants having medicinal properties and which have beneficial properties in relation to weight control. The compositions of the present invention are prepared using concepts of MCM.

Accordingly, one aspect of the present invention is directed to a composition comprising an extract of herbs or horticultural or botanical equivalents of herbs or chemical or functional equivalents of the herbal extract wherein said composition is effective in the treatment or prophylaxis of obesity.

The preferred herbs in accordance with the present invention are those which:

(i) are useful for treating disorders affecting lipid metabolism;

(ii) have warming properties and exhibit tonic properties;

(iii) are effective in reducing blood sugar levels;

(iv) are useful in treating coronary heart disease;

(v) exhibit sedative properties; and/or

(vi) promote increased cholesterol excretion.

Accordingly, another aspect of the present invention contemplates a composition comprising two or more herbs or horticultural or botanical equivalents of said herbs wherein said herbs are selected from those which:

(i) are useful for treating disorders affecting lipid metabolism;

(ii) have warming properties and exhibit tonic properties;

(iii) are effective in reducing blood sugar levels;

(iv) are useful in treating coronary heart disease;

(v) exhibit sedative properties; and/or

(vi) promote increased cholesterol excretion; or chemical or functional equivalents of said herbal extract wherein said composition is effective in the treatment or prophylaxis of obesity.

Preferably, the composition comprises at least about three, more preferably at least about four, even more preferably at least about five, still more preferably at least about six, even still more preferably at least about seven and yet even still more preferably at least about eight or more herbs or their horticultural or botanical equivalents.

In a particularly preferred embodiment, the composition comprises extracts from herbs selected from two or more of Polygonatum sp, Nephelium sp, Hordeum sp, Ligusticum sp, Lilium sp, Rhizoma sp, Gynura sp, Companumaea sp, Codonopsis sp, and fruit of a tree, ginger essence and Juniper Q.

Accordingly, another aspect of the present invention provides a composition comprising two or more herbs or extracts of two or more herbs selected from species selected from Polygonatum sp, Nephelium sp, Hordeum sp, Ligusticum sp, Lilium sp, Rhizoma sp, Gynura sp, Companumaea sp, Codonopsis sp, and fruit of a tree, ginger essence and Juniper Q and which herbs:

(i) are useful for treating disorders affecting lipid metabolism;

(ii) have warming properties and exhibit tonic properties;

(iii) are effective in reducing blood sugar levels;

(iv) are useful in treating coronary heart disease;

(v) exhibit sedative properties; and/or

(vi) promote increased cholesterol excretion; or horticulture or botanical equivalent of the herb or chemical or functional equivalent of the herbal extract wherein said composition is useful in the treatment or prophylaxis of obesity.

The most preferred herbs in accordance with the present invention are selected from two or more of Gynura pinnatifida root, Lilium brownii bulb, Nephilium longana fruit, Polygonatum multiforum root, Polygonatum canaliculatum, Condonopsis lanceolata root, Ligusticum sinense root, Hordeum vulgare, Ligusticum chuanxiong, Rhizoma zingiberis, Campanumaea lanceolata, Apple green, ginger essence and Juniper Q.

Accordingly, the present invention provides a composition useful in the treatment and/or prophylaxis of obesity and other forms of weight control, said composition comprising two or more herbs or botanical or horticultural equivalents thereof, said herbs selected from Gynura pinnatifida root, Lilium brownie bulb, Nephilium longana fruit, Polygonatum multiforum root, Polygonatum canaliculatum, Condonopsis lanceolata root, Ligusticum sinense root, Hordeum vulgare, Ligusticum chuanxiong, Rhizoma zingiberis, Campanumaea lanceolata, Apple green, ginger essence and Juniper Q.

The present invention extends to any or all of the above-mentioned herbs or botanical or horticultural equivalents thereof as well as chemical or functional equivalents of the extracts prepared from the herbs. The present invention is not necessarily limited, however, to the major constituents of the herbs since efficacious amounts of a combination of trace elements or constituents of the above-mentioned herbs may also be required. A botanical or horticultural equivalent of a herb is a plant which is physiologically, biochemically, immunologically, structurally or functionally similar to a herb or herbal extract or which exhibits therapeutically similar properties. For example, many Australian native plants have medicinal value which may be used to supplement or substitute for a herb.

The composition of the present invention are prepared on the basis of a rationale approach. Generally, the compositions comprise from about 0.1% wt./vol. to about 40% wt./vol. dry weight of herbs or extracts thereof. Preferably, the dry weight of herbs or extract is from about 1% wt./vol. to about 20% wt./vol.

The compositions of the present invention may also comprise one or more pharmaceutically acceptable carriers and/or diluents. Such carriers and/or diluents include any and all solvents, water, dispersion media, coatings, antibacterial and antifungal agents, isotonic and absorption delaying agents and the like. Preferably, the diluent is water or a water or alcohol based extract of the herb.

The composition according to the present invention may be formulated for administration by any conventional route such as oral, rectal, nasal, topical (dermal), or via aerosol parental. Thus the composition may be in the form of tablets, capsules, suspensions, emulsions, solutions, suppositories, sprays or injectibles. Formulations for oral use include tablets which contain the active ingredient in admixture with pharmaceutically acceptable inert excipients. These excipients may be, for example, inert diluents such as calcium carbonate, sodium chloride, lactose, calcium phosphate, sodium phosphate, etc.; granulating and disintegrating agents, for example, potato starch, alginic acid, and the like; binding agents, for example, starch, gelatin or acacia, and the like; and lubricating agents, for example, magnesium stearate, stearic acid or talc. Other pharmaceutically acceptable excipients can be colorants, flavoring agents, plasticizers, humectants, and the later. The tablets may be uncoated or they may be coated by known techniques.

Alternatively, the active ingredients of the weight reduction formulation may be delivered over an extended time period by delaying disintegration and absorption in the gastrointestinal tract to provide a sustained release effect. For example, a time delay material such as glyceral monostearate or glycerol distearate may be employed. The active ingredient may be placed in an extended release dosage form by techniques which are well known in the art (see, e.g., Baker and Richard, Controlled Release of Biologically Active Agents, John Wiley and Sons, New York, 1986). The inactive excipients in a tablet may include calcium carbonate, dicalcium phosphate, microcrystalline cellulose, croscarmellose sodium, stearic acid, silica, magnesium stearate and pharmaceutical glaze. Alternatively, the active ingredient or extract thereof may be delivered in a soft or hard gel capsule by mixing the active ingredient with water or an oil medium such as peanut oil, liquid paraffin, or olive oil and enclosing it within the capsule or gel capsule.

The dosage may also be administered as an oral liquid dosage form by suspending the active ingredients or extracts thereof in an aqueous solution in admixture with dispersing or wetting agent, suspending agent and one or more preservatives. Suitable dispersing or wetting agents are, for example, naturally occurring phosphatides, for example, lecithin, or condensation products of ethylene oxide, fatty acids, long chain aliphatic acids, or a partial ester derived from fatty acids and a hexitol or hexitol anhydrides, for example, polyoxyethylene stearate, polyoxyethylene sorbitol monooleate, polyoxyethylene sorbitan monooleate, etc. Suitable suspending agents are, for example, sodium carboxymethylcellulose, methylcellulose, sodium alginate, etc.

The pharmaceutical formulation may also be delivered parenterally in dosage forms containing conventional pharmaceutically acceptable carriers and adjuvants. The formulation and preparation of such compositions is well known to those skilled in the art. For example, specific formulations can be found in the text Remington's Pharmaceutical Sciences (Mack Publishing Co., Easton, Pa., 1995). The active ingredients may be dissolved in vehicles such as water, isotonic sodium hydroxide solutions, etc. The aqueous formulations may also contain a preservative such as methyl, ethyl or n-propyl p-hydroxybenzoate.

To prepare the weight loss and appetite suppressant of the present invention in a tablet form, the extract of the two herbs were combined with excipients in an appropriate ratio and thereafter tableted by methods which are well known in the art (see, e.g., Remington's Pharmaceutical Sciences, ibid.)

In a particularly preferred embodiment, the herbs are contained and subjected to conventional extraction techniques. Such techniques include water extraction, solvent extraction, alcohol extraction and/or acid or base extraction. The resulting extract is then preferably incorporated into a capsule.

The compositions of the present invention are conveniently defined on the basis of, for example, a spectral or chromatographic profile. The profile may be of the composition as a whole or of the individual components. In relation to the latter, a composition may be considered a formulation of three or more components in a particular ratio. Furthermore, a maximum tolerated dose may be obtained for each component or each herbal extract and a composition prepared on the basis of a maximum amount of herbal components tolerable by a subject.

Accordingly, the present invention provides a composition comprising maximum tolerated doses of herbs or extracts of herbs or horticultural or botanical equivalents of said herbs wherein said herbs are selected from Gynura pinnatifida root, Lilium brownii bulb, Nephilium Longana fruit, Polygonatum multiforum root, Polygonatum canaliculatum, Condonopsis lanceolata root, Ligusticum sinense root, Hordeum vulgare, Ligusticum chuanxiong, Rhizoma zingiberis, Campanumaea lanceolata, Apple green, ginger essence and Juniper Q wherein said composition is useful for the treatment or prophylaxis of obesity.

The herbs of the present invention are conveniently defined in terms of a fingerprints of, for example, nucleic acids (e.g., DNA), total protein, carbohydrate profile and immunological profile. This ensures quality control and consistency of preparation.

The term “obesity” is used in its broadest term and includes conditions involving at least about 2% or more excess body weight compared to normal controls. The composition may be used in a treatment sense meaning that it is administered to obese subjects or it may be used in a preventative or prophylactic sense to subjects desirous of maintaining a non-obese condition. The term “obese” includes being overweight and the treatment and/or prophylaxis of obesity includes all elements of weight control.

Another aspect of the present invention contemplates a method for the treatment of prophylaxis of obesity in a subject, said method comprising administering to said subject an effective amount of a composition comprising two or more herbs or horticultural or botanical equivalents of said herbs wherein said herbs are selected from those which:

(i) are useful for treating disorders affecting lipid metabolism;

(ii) have warming properties and exhibit tonic properties;

(iii) are effective in reducing blood sugar levels;

(iv) are useful in treating coronary heart disease;

(v) exhibit sedative properties; and/or

(vi) promote increased cholesterol excretion or chemical or functional equivalents of said herbal extract

More particularly, the composition comprising two or more herbs or extracts of two or more herbs selected from species selected from Polygonatum sp, Nephelium sp, Hordeum sp, Ligusticum sp, Lilium sp, Rhizoma sp, Gynura sp and Codonopsis sp. and which herbs:

(i) are useful for treating disorders affecting lipid metabolism;

(ii) have warming properties and exhibit tonic properties;

(iii) are effective in reducing blood sugar levels;

(iv) are useful in treating coronary heart disease;

(v) exhibit sedative properties; and/or

(vi) promote increased cholesterol excretion, or horticulture or botanical equivalent of the herb or chemical or functional equivalent of the herbal extract.

Even more particularly, the composition comprising two or more herbs or botanical or horticultural equivalents thereof, said herbs selected from Gynura pinnatifida root, Lilium brownie bulb, Nephilium longana fruit, Polygonatum multiforum root, Polygonatum canaliculatum, Condonopsis lanceolata root, Ligusticum sinense root, Hordeum vulgare, Ligusticum chuanxiong, Rhizoma zingiberis, Campanumaea lanceolata, Apple green, ginger essence and Juniper Q.

The present invention extends to any or all of the above-mentioned herbs or botanical or horticultural equivalents thereof as well as chemical or functional equivalents of the extracts prepared from the herbs. The present invention is not necessarily limited, however, to the major constituents of the herbs since efficacious amounts of a combination oftrace elements or constituents of the above-mentioned herbs may also be required. A botanical or horticultural equivalent of a herb is a plant which is physiologically, biochemically, immunologically, structurally or functionally similar to a herb or herbal extract or which exhibits therapeutically similar properties. For example, many Australian native plants have medicinal value which may be used to supplement or substitute for a herb.

The composition of the present invention are prepared on the basis of a rationale approach. Generally, the compositions comprise from about 0. 1% wt./vol. to about 40% wt./vol. dry weight of herbs or extracts thereof. Preferably, the dry weight of herbs or extract is from about 1% wt./vol. to about 20% wt./vol.

The compositions of the present invention may also comprise one or more pharmaceutically acceptable carriers and/or diluents. Such carriers and/or diluents include any and all solvents, water, dispersion media, coatings, antibacterial and antifungal agents, isotonic and absorption delaying agents and the like. Preferably, the diluent is water or a water or alcohol based extract.

Yet another aspect of the present invention provides a therapeutic pack comprising dried herbs mixed together or in separate compartments and which are admixed with water or other solvent prior to use wherein said herbs are selected from two or more of Polygonatum sp, Nephelium sp, Hordeum sp, Ligusticum sp, Lilium sp, Rhizoma sp, Gynura sp, Companumaea sp, Codonopsis sp, and fruit of a tree, ginger essence and Juniper Q.

Preferably, the herbs are selected from two or more of Gynura pinnatifida root, Lilium brown bulb, Nephilium longana fruit, Polygonatum multiforum root, Polygonatum canaliculatum, Condonopsis lanceolata root, Ligusticum sinense root, Hordeum vulgare, Ligusticum chuanxiong, Rhizoma zingiberis, Campanumaea lanceolata, Apple green, ginger essence and Juniper Q.

The effective amount is the amount which ameliorates the effects of obesity. Conveniently, the compositions are administered in liquid form such as admixing the composition in water and administered prior to or after meals.

The present invention does not extend to the following compositions at the concentrations listed per ml:

| | | |
|---|---|---|
| Gynura pinnatifida | (50 | μg) |
| Lilium brownii | (0.5 | mg) |
| Hordeum vulgare | (750 | μg) |
| Nephelium longana | (0.1 | mg) |
| Polygonatum canaliculatum | (50 | μg) |
| Campanumaea lanceolata | (50 | μg) |
| Ligusticum sinensis | (250 | μg) |
| Apple green | (10 | mg) |
| Ginger essence | (50 | mg) |
| Juniper Q | (25 | μg) |

The present invention also does not extend to the following compositions at the concentrations listed per 270 μL:

| | | |
|---|---|---|
| Gynura pinnatifida root | (13.5 | μg) |
| Lilium brownii bulb | (140 | μg) |
| Nephelium longana fruit | (30 | μg) |
| Polygonatum multiforum root | (13.5 | μg) |
| Condonopsis lanceolata root | (13.5 | μg) |
| Ligusticum sinense root | (67.5 | μg) |

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Extraction and Formulation

Herbal extraction techniques were performed according to standard protocols and were designed to maintain maximal levels of active components.

### Example 2

### Obesity Control

The conventional method of counting calorie intake often fails to be effective in treatment of obesity especially obesity associated with Type 3 fat.

Type 3 fat is a gel like fat with water and waste trapped in an immoveable pocket below the skin. These pockets of Type 3 fat are like sponges that can absorb water, then expand, causing the ripples, bulges and flabbiness of the obese.

Because this form of fat is not the normal structural type of fat, even the most carefully controlled and stringent diet often fails to correct the problem.

When the normal intake of calories are reduced, the body automatically triggers the mechanism to utilize its own stored reserves of food, i.e., fat.

On a low calorie diet, many parts of the body will demonstrate the visual effect of this utilization of the body's reserves of food and some slimming or weight loss may occur. But the Type 3 fat will remain. It is not consumed like normal fat.

Obesity control should therefore aim at not only a low calorie intake, but also the mobilization of Type 3 fats.

This can be achieved via acupuncture for control of the calorie intake, through hunger suppression, and the use of a herbaceous substance known to be effective in the mobilization of Type 3 fat.

Slimax™ is the trade name of a Chinese Herbal formulation comprising the following agents:

| | | |
|---|---|---|
| Lilium brownii | 100 | grams |
| Hordeum vulgari | 150 | grams |
| Nephelium longana | 20 | grams |
| Canaliculatum | 10 | grams |
| Ligusticum | 50 | grams |

The herbs are extracted with 70% alcohol, and heated until all the alcohol is removed. The residue is reconstituted to 100 mls. This is used in the experiments.

### Rats

Four lots of D.A. female rats (in-bred), in groups of six were used. The rats were 10 weeks old.

Group A Control Group

Six rats acting as a control, fed on normal basal diet.

Group B Slimax Group

Six rats were given a basal diet plus Slimas™ added to their drinks. The Slimax was given in a one per hundred part of water.

The experiment was carried out on a double blind basis.

The rats were weighed each day at the same time and by the same scale (Quadrabeam Mod. 483). The amounts of food and water consumed were recorded and urine volume was recorded at the same time.

The rats were housed in a temperature, humidity, noise and dust controlled area.

At the end of six weeks the rats were sacrificed and carcased, then analyzed for moisture, fat, nitrogen and ash.

The food intake per gram of Rat per day (Table 1) is the highest in the control group A being 12.7 grams.

TABLE 1

| | | Feed intake per gram, per |
|---|---|---|
| | | head per gram |
| Group A | No acupuncture | 12.7 g |
| | No Slimax ™ | |
| | (control) | |
| Group B | Acupuncture | 11.6 g |
| | No Slimax ™ | |
| Group C | No acupuncture | 11.4 g |
| | Slimax ™ | |
| Group D | Acupuncture | 10.4 g |
| | Slimax ™ | |

The Slimax ™ Group C showed a decrease of food intake. The decrease in this case is likely due to the mobilization and metabolization of Type 3 fat supplying the extra energy for the body and decreasing the body's need for food.
Group A - There is a steady and gradual weight increase in the weight of the group as expected in a normal free diet situation. The rate of energy expenditure is less than the calorie consumption. The excess calories show here as weight increase.
Group C - The Slimax ™ group C started with a steady weight loss until the fifth week when a marked decrease in weight occurs. This may be due to the Slimax ™ taking time to work on the excess fat. This excess fat takes time to be utilized by the body, but once the process is started, the reduction is rapid.

### Example 3

### Herbal Plants

Herbal and other medicinal plants are subjected to molecular characterization to provide a fingerprint to facilitate consistency of supply and quality control. Finger prints are prepared using nucleic acid or protein chromatography or immunological interactivity.

### Example 4

### Maximizing Effects of Herbal Extracts

The maximum tolerated dose is determined for each of the components of the herbal composition and the concentrations manipulated to provide the most efficacy result in terms of weight control.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprising an extract of herbs or horticultural or botanical equivalents of herbs or chemical or functional equivalents of the herbal extract wherein said composition is effective in the treatment or prophylaxis of obesity.

2. The composition of claim 1, comprising at least three herbs or horticultural or botanical equivalents of said herbs wherein said herbs are selected from those which:
(i) are useful for treating disorders affecting lipid metabolism;
(ii) have warming properties and exhibit tonic properties;
(iii) are effective in reducing blood sugar levels;
(iv) are useful in treating coronary heart disease;
(v) exhibit sedative properties; or
(vi) promote increased cholesterol excretion,
or chemical or functional equivalents of said herbal extract.

3. The composition of claim 2, comprising at least three herbs or extracts of said herbs, wherein said herbs are selected from the group consisting of Polygonatum sp, Nephelium sp, Hordeum sp, Ligusticum sp, Lilium sp, Rhizoma sp, Gynura sp, Companumaea sp, Codonopsis sp, fruit of a tree, ginger essence and Juniper Q, wherein said herbs:
(i) are useful for treating disorders affecting lipid metabolism;
(ii) have warming properties and exhibit tonic properties;
(iii) are effective in reducing blood sugar levels;
(iv) are useful in treating coronary heart disease;
(v) exhibit sedative properties; or
(vi) promote increased cholesterol excretion,
or a horticulture or a botanical equivalent of said herbs or a chemical or functional equivalent of said extracts.

4. The composition of claim 3, comprising at least three herbs or botanical or horticultural equivalents thereof, said herbs selected from the group consisting of Gynura pinnatifida root, Lilium brownii bulb, Nephilium longana fruit, Polygonatum multiforum root, Polygonatum canaliculatum, Condonopsis lanceolata root, Ligusticum sinense root, Hordeum vulgare, Ligusticum chuanxiong, Rhizoma zingiberis, Campanumaea lanceolata, Apple green, ginger essence and Juniper Q.

5. The composition of claim 1, comprising maximum tolerated doses of herbs or extracts of herbs or horticultural or botanical equivalents of said herbs wherein said herbs are selected from the group consisting of Gynura pinnatifida root, Lilium brownie bulb, Nephilium longana fruit, Polygonatum multiforum root, Polygonatum canaliculatum, Condonopsis lanceolata root, Ligusticum sinense root, Hordeum vulgare, Ligusticum chuanxiong, Rhizoma zingiberis, Campanumaea lanceolata, Apple green, ginger essence and Juniper Q.

6. The composition of claim 1, comprising an extract of herbs or horticultural or botanical equivalent of herbs or chemical or functional equivalents of the herbal extract wherein said composition comprises:

| | |
|---|---|
| about 50 μg | Gynura pinnatifida; |
| about 0.5 mg | Lilium brownii; |
| about 750 μg | Hordeum vulgare; |
| about 0.1 mg | Nephelium longana; |
| about 50 μg | Polygonatum canaliculatum, |
| about 50 μg | Campanumaea lanceolata; |
| about 250 μg | Ligusticum sinensis; |
| about 10 mg | Apple green; |
| about 50 mg | Ginger essence; and |
| about 25 μg | Juniper Q. |

7. The composition of claim 1, wherein said composition comprises:

| | | |
|---|---|---|
| Gynura pinnatifida root | 13.5 | μg; |
| Lilium brownii bulb | 140 | mg; |
| Nephelium longana fruit | 30 | mg; |
| Polygonatum multiforum root | 13.5 | μg; |
| Condonopsis lanceolata root | 13.5 | μg; and |
| Ligusticum sinensis root | 67.5 | μg. |

8. A method for the treatment or prophylaxis of obesity in a subject, said method comprising administering to said subject a composition comprising at least three herbs or horticultural or botanical equivalents of said herbs, or chemical or functional equivalents of an extract of said herbs, wherein said herbs are selected from those which:
(i) are useful for treating disorders affecting lipid metabolism;
(ii) have warming properties and exhibit tonic properties;
(iii) are effective in reducing blood sugar levels;
(iv) are useful in treating coronary heart disease;
(v) exhibit sedative properties; and/or
(vi) promote increased cholesterol excretion,
in an amount and for a time effective for the treatment or prophylaxis of said obesity in said subject.

9. The method of claim 8, wherein said subject is a human subject.

10. The method of claim 8, wherein said composition comprises at least three herbs or extracts of at least three herbs selected from species selected from Polygonatum sp, Nephelium sp, Hordeum sp, Ligusticum sp, Lilium sp, Rhizoma sp, Gynura sp, Companumaea sp, Codonopsis sp, and fruit of a tree, ginger essence and Juniper Q; or horticulture or botanical equivalent of the herb or chemical or functional equivalents of said extract of said herbs, wherein said herbs:
(i) are useful for treating disorders affecting lipid metabolism;
(ii) have warming properties and exhibit tonic properties;
(iii) are effective in reducing blood sugar levels;
(iv) are useful in treating coronary heart disease;
(v) exhibit sedative properties; or
(vi) promote increased cholesterol excretion.

11. The method of claim 10, wherein the composition comprises at least three herbs or botanical or horticultural equivalents thereof, said herbs being selected from the group consisting of Gynura pinnatifida root, Lilium brownii bulb, Nephilium Longana fruit, Polygonatum multiforum root, Polygonatum canaliculatum, Condonopsis lanceolata root, Ligusticum sinense root, Hordeum vulgare, Ligusticum chuanxiong, Rhizoma zingiberis, Campanumaea lanceolata, Apple green, ginger essence and Juniper Q.

12. The method of claim 11, wherein said composition comprises:

| | | |
|---|---|---|
| Gynura pinnatifida | 50 | μg; |
| Lilium brownii | 0.5 | mg; |
| Hordeum vulgare | 750 | μg; |
| Nephelium longana | 0.1 | mg; |
| Polygonatum canaliculatum | 50 | μg; |
| Campanumaea lanceolata | 50 | μg; |
| Ligusticum sinensis | 250 | μg; |
| Apple green | 10 | mg; |
| Ginger essence | 50 | mg; and |
| Juniper Q | 25 | μg. |

13. The method of claim 11, wherein said composition comprises:

| | | |
|---|---|---|
| Gynura pinnatifida root | 13.5 | μg; |
| Lilium brownii bulb | 140 | mg; |
| Nephelium longana fruit | 30 | mg; |
| Polygonatum multiforum root | 13.5 | μg; |
| Condonopsis lanceolata root | 13.5 | μg; and |
| Ligusticum sinensis root | 67.5 | μg. |
